(19) United States
(12) Patent Application Publication
(10) Pub. No.: US 2026/0199324 A1
(43) Pub. Date: Jul. 16, 2026

(54) METHOD OF TREATING BREAST CANCER

(71) Applicants: Arvinas Operations, Inc., New Haven, CT (US); Pfizer Inc., New York, NY (US)

(72) Inventors: Ronald Peck, Cheshire, CT (US); Derek Zhiye Yang, San Diego, CA (US); Weiwei Tan, San Diego, CA (US)

(21) Appl. No.: 19/134,632

(22) PCT Filed: Dec. 1, 2023
(86) PCT No.: PCT/US2023/082089

§ 371 Date: May 30, 2025

Related U.S. Application Data
(60) Provisional application No. 63/429,836, filed on Dec. 2, 2022.
(60) Provisional application No. 63/603,606, filed on Nov. 28, 2023.

(51) Int. Cl. A61K 31/496; A61K 31/4439

(52) U.S. Cl. CPC A61K 31/496; A61K 31/4439

(57) ABSTRACT

The invention relates to a method for treating cancer in a subject receiving a substrate of P-glycoprotein comprising administering Compound A having a structure of: or a pharmaceutically acceptable salt thereof, to the subject and monitoring the subject for adverse reactions during treatment with Compound A, or a pharmaceutically acceptable salt thereof.

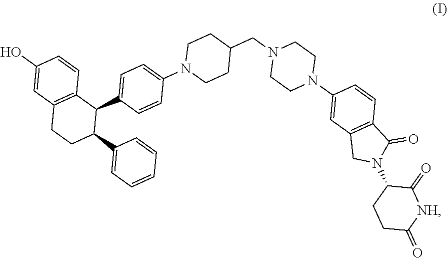 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

### CROSS-REFERENCE TO RELATED APPLICATIONS

This application claims priority to, and the benefit of, U.S. Provisional Application No. 63/429,836, filed Dec. 2, 2022, and U.S. Provisional Application No. 63/603,606, filed Nov. 28, 2023, the contents of each of which are incorporated by reference in their entireties for all purposes.

### BACKGROUND OF THE DISCLOSURE

Certain bifunctional compounds can target specific cellular proteins for degradation via the ubiquitin-proteasome system. Examples of such proteolysis targeting chimeric compounds (i.e., “PROTAC® protein degraders”) that target the Estrogen Receptor (ER) for ubiquitination and subsequent degradation are disclosed in International Publication No. WO 2018/102725, which is incorporated herein by reference in its entirety. Such bifunctional molecules exhibit a range of pharmacological activities consistent with the degradation of the ER including, but not limited to, treatment or amelioration of a disease condition such as cancer (e.g., breast cancer, uterine cancer, ovarian cancer, prostate cancer, endometrial cancer), or endometriosis.

A bifunctional molecule of particular interest is(S)-3-(5-(4-((1-(4-((1R,2S)-6-hydroxy-2-phenyl-1,2,3,4-tetrahydronaphthalen-1-yl)phenyl) piperidin-4-yl)methyl) piperazin-1-yl)-1-oxoisoindolin-2-yl) piperidine-2,6-dione or (35)-3-[1,3-dihydro-1-oxo-5-[4-[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (referred to herein as “Compound A”), which has the molecular formula of C₄₅H₄₉N₅O₄ and the following structure:

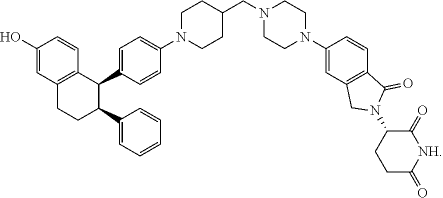 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is under development as a PROTAC® protein degrader that targets ER for the potential treatment of breast cancer and has been shown to be a useful modulator of targeted protein ubiquitination and degradation via the ubiquitin-proteasome pathway.

Compound A may be administered to cancer patients that are also being administered a concomitant medication. Concomitant medications include, e.g., other prescription medications or over-the-counter drugs that a cancer patient might take in addition to Compound A. Concomitant medications may be used by a patient for the same indication or for other indications.

The simultaneous or nearly simultaneous (e.g., concomitant) presence of two drugs in a subject may alter the effects of one or the other, or both, drugs. Such alterations are termed drug-drug interactions. Concomitant administration or coadministration of two or more drugs mean that the drugs are present in the body at the same time. Concomitant administration or coadministration of different drugs often leads to adverse effects since the metabolism and/or excretion of each drug may reduce or interfere with the metabolism and/or excretion of the other drug(s), thus increasing the effective concentrations of those drugs as compared to effective concentrations of those drugs when administered alone.

Accordingly, methods of treatment that address and/or minimize drug-drug interactions in the treatment of cancer are needed.

### SUMMARY OF THE INVENTION

The present disclosure provides, in part, methods for administering Compound A, or a pharmaceutically acceptable salt thereof, to a subject, for treating cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Provided herein are methods for treating cancer in a subject receiving a substrate of P-glycoprotein comprising administering Compound A having a structure of:

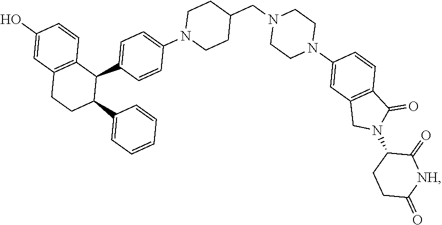 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, to the subject and monitoring the subject for adverse reactions during treatment with Compound A, or a pharmaceutically acceptable salt thereof.

Provided herein are methods for treating cancer in a subject receiving dabigatran etexilate mesylate comprising administering Compound A having a structure of:

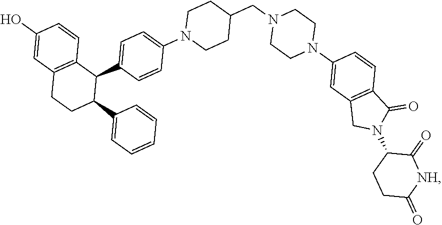 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, to the subject and monitoring the subject for adverse reactions during treatment with Compound A, or a pharmaceutically acceptable salt thereof.

Provided herein are methods for treating cancer comprising administering to a subject Compound A having a structure of:

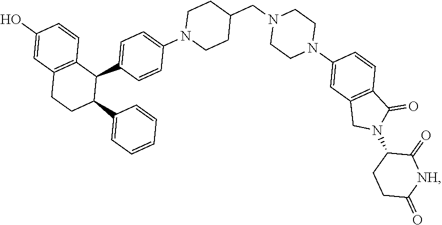 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, wherein Compound A is not to be administered with concomitant administration of a substrate of P-glycoprotein.

Provided herein are methods for treating cancer comprising administering to a subject Compound A having a structure of:

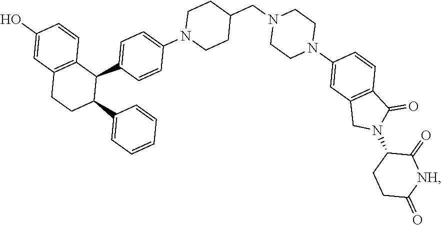 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, wherein Compound A is not to be administered with concomitant administration of dabigatran.

Additionally provided herein are methods for treating cancer comprising administering Compound A having a structure of:

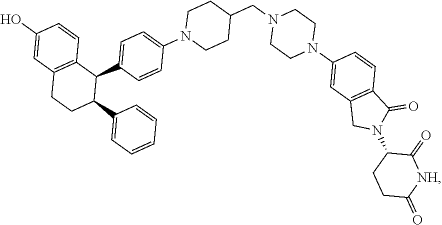 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, to a subject, wherein a physician is provided with instructions to monitor the subject for adverse reactions during concomitant administration of Compound A, or a pharmaceutically acceptable salt thereof, and a substrate of P-glycoprotein.

Additionally provided herein are methods for treating cancer comprising administering Compound A having a structure of:

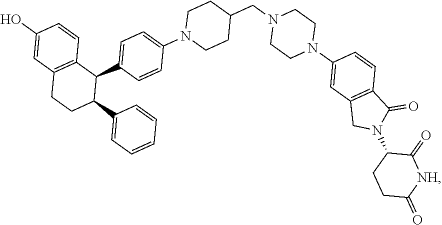 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, to a subject, wherein a physician is provided with instructions to monitor the subject for adverse reactions during concomitant administration of Compound A, or a pharmaceutically acceptable salt thereof, and dabigatran etexilate mesylate.

Additionally provided herein are methods for treating cancer comprising administering to a subject Compound A having a structure of:

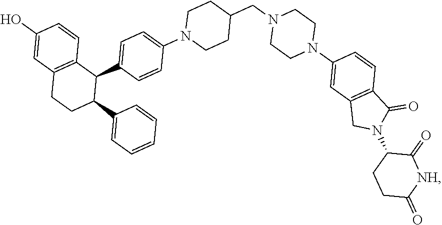 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, wherein a physician is provided with instructions including a warning that the treatment is not to be administered with concomitant administration of a substrate of P-glycoprotein.

Additionally provided herein are methods for treating cancer comprising administering to a subject Compound A having a structure of:

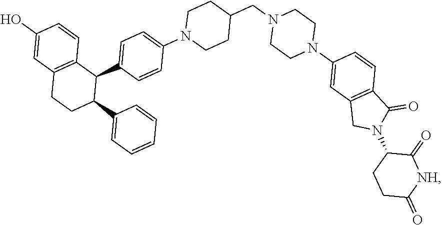 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, wherein a physician is provided with instructions including a warning that the treatment is not to be administered with concomitant administration of dabigatran etexilate mesylate.

In embodiments, the adverse reactions are selected from the group consisting of diarrhea, nausea, dry skin, hot flush, and headache.

In embodiments, a daily dose of Compound A, or a pharmaceutically acceptable salt thereof, is administered to the subject.

In embodiments, a daily dose of Compound A, or a pharmaceutically acceptable salt thereof, is administered to the subject and the daily dose of Compound A, or a pharmaceutically acceptable salt thereof, is 200 mg.

In embodiments, a daily dose of Compound A, or a pharmaceutically acceptable salt thereof, is administered to the subject and the daily dose of Compound A, or a pharmaceutically acceptable salt thereof, is 100 mg.

In embodiments, a daily dose of Compound A, or a pharmaceutically acceptable salt thereof, is administered to the subject and the daily dose of Compound A, or a pharmaceutically acceptable salt thereof, is administered once per day (QD).

In embodiments, a daily dose of Compound A, or a pharmaceutically acceptable salt thereof, is administered to the subject and the daily dose of Compound A, or a pharmaceutically acceptable salt thereof, is administered orally to the subject.

In embodiments, the subject is in a fed state.

In embodiments, the daily dose of Compound A, or a pharmaceutically acceptable salt thereof, is not to be administered with concomitant administration of substrate of P-glycoprotein.

In embodiments, the substrate of P-glycoprotein is apixaban, colchicine, cyclosporine, dabigatran etexilate mesylate, digoxin, edoxaban, fexofenadine, rivaroxaban, tacrolimus, or talinolol.

In embodiments, the substrate of P-glycoprotein is dabigatran etexilate mesylate.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer. In embodiments, the cancer is breast cancer.

In embodiments, the breast cancer is metastatic or locally advanced. In embodiments, the breast cancer is estrogen receptor positive (ER+) breast cancer.

In embodiments, the estrogen receptor positive (ER+) breast cancer is human epidermal growth factor receptor 2 negative (HER2−).

In embodiments, the subject is human.

It is to be understood that both the foregoing general description and the following detailed description are exemplary and explanatory only and are not restrictive of the invention, as claimed.

### DETAILED DESCRIPTION

The present invention may be understood more readily by reference to the following detailed description of the embodiments of the invention and the Examples included herein. It is to be also understood that the terminology used herein is for the purpose of describing specific embodiments only and is not intended to be limiting.

A P-glycoprotein (P-gp) substrate is a substance that uses the P-glycoprotein transporter for various activities, including drug absorption, drug excretion, and other important activities which can lead to changes in the body or changes in the effects of other drugs on the body (Lin J H, Yamazaki M: Role of P-glycoprotein in pharmacokinetics: clinical implications, Clin Pharmacokinet. 2003; 42(1):59-98). Without wishing to be bound by theory, administration of a P-gp substrate with a P-gp inhibitor, or compounds that act as a P-gp inhibitor, can lead to increased plasma exposures of the P-gp substrate.

Accordingly, provided herein are methods for treating cancer in a subject receiving a substrate of P-glycoprotein comprising administering Compound A having a structure of:

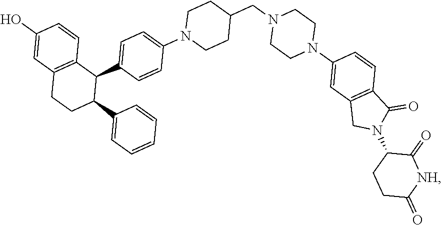 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, to the subject and monitoring the subject for adverse reactions during treatment with Compound A, or a pharmaceutically acceptable salt thereof.

### Definitions

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

As used herein, the singular form “a,” “an,” and “the” include plural references unless indicated otherwise. For example, “a” substituent includes one or more substituents.

As used herein, the term “about” when used to modify a numerically defined parameter (e.g., the dose of Compound A) means that the parameter may vary by as much as 10% below or above the stated numerical value for that parameter. For example, a dose of about 200 mg means 200 mg±10%, i.e., it may vary between 180 mg and 220 mg.

As used herein, terms, including, but not limited to, “agent”, “compound”, “drug”, “medication” and “therapeutic agent” may be used interchangeably to refer to compounds included in the present disclosure and methods and uses of the present invention, specifically Compound A.

Compound A, having the structure:

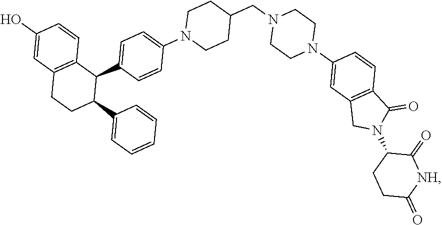 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

and pharmaceutically acceptable salts thereof, are disclosed in International Publication No. WO 2018/102725 and U.S. Pat. Nos. 10,647,698, 10,899,742 and 11,104,666; International Publication No. WO 2021/041348; U.S. Ser. No. 17/472,847; U.S. Ser. No. 17/548,842; and U.S. Ser. No. 17/873,748. The contents of each of the foregoing references are incorporated herein by reference in their entireties.

Compound A is a Biopharmaceutics Classification System Class IV compound (low solubility/low permeability). Compound A may interconvert to its epimer, Compound B:

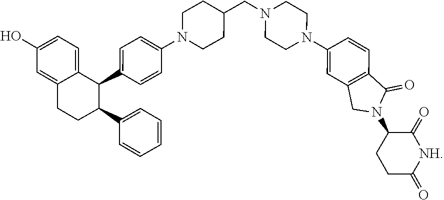 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Preclinical data demonstrates that the exposure of Compound B is limited compared to Compound A (<26%). Without wishing to be bound by theory, evidence indicates that Compound B does not degrade the ER; however, Compound B shows similar antagonism of ER-dependent transcription compared to Compound A.

In embodiments, the pharmaceutically acceptable salts of compounds are described herein. Pharmaceutically acceptable salts of the compounds described herein include the acid addition and base addition salts thereof.

In embodiments, the pharmaceutically acceptable acid addition salts of the compounds are described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine, and zinc salts.

The compounds described herein that are basic in nature are capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium, and other base salts of pharmaceutically acceptable organic amines.

Hemisalts of acids and bases may also be formed, for example, hemisulphate, and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

Dabigatran etexilate (β-Alanine, N-[[2-[[[4-[[[(hexyloxy) carbonyl]amino]iminomethyl] phenyl]amino]methyl]-1-methyl-1H-benzimidazol-5-yl]carbonyl]-N-2-pyridinyl-, ethyl ester) is oral prodrug of dabigatran (3-[[2-[(4-carbamimidoylanilino) methyl]-1-methylbenzimidazole-5-carbonyl]-pyridin-2-ylamino]propanoic acid), a direct thrombin inhibitor. Dabigatran etexilate mesylate is an anticoagulant is used to treat and prevent blood clots and to prevent stroke in people with atrial fibrillation. In some embodiments, dabigatran etexilate mesylate is marketed as Pradaxa®. The structures of dabigatran and dabigatran etexilate mesylate are shown below:

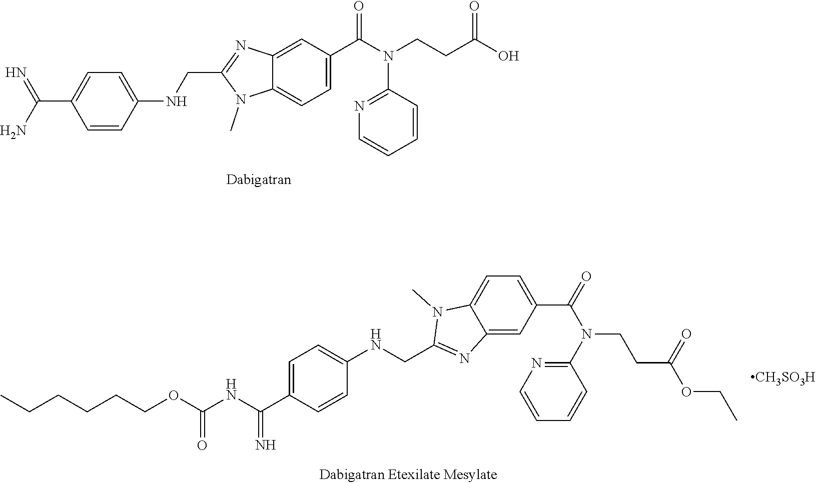

Dabigatran etexilate mesylate is an oral, reversible, competitive, and direct thrombin inhibitor and is an in vitro and in vivo substrate of P-glycoprotein (P-gp). Dabigatran etexilate mesylate may be used as a probe for intestinal P-gp inhibition and has been recommended as a probe for that purpose by regulatory agencies.

Additionally, P-gp substrates include apixaban, colchicine, cyclosporine, digoxin, edoxaban, fexofenadine rivaroxaban, tacrolimus, and talinolol.

### Methods of Administration

Provided herein are methods for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof.

Provided herein are methods for treating cancer comprising administering to a subject Compound A, or a pharmaceutically acceptable salt thereof, wherein Compound A, or a pharmaceutically acceptable salt thereof, is not to be administered with concomitant administration of a substrate of P-glycoprotein.

Provided herein are methods for treating cancer comprising administering to a subject Compound A, or a pharmaceutically acceptable salt thereof, wherein Compound A, or a pharmaceutically acceptable salt thereof, is not to be administered with concomitant administration of dabigatran etexilate mesylate.

Additionally provided herein are methods for treating cancer comprising administering Compound A, or a pharmaceutically acceptable salt thereof, to a subject, wherein a physician is provided with instructions to monitor the subject for adverse reactions during concomitant administration of Compound A, or a pharmaceutically acceptable salt thereof, and a substrate of P-glycoprotein.

Additionally provided herein are methods for treating cancer comprising administering to a subject Compound A, or a pharmaceutically acceptable salt thereof, wherein a physician is provided with instructions including a warning that the treatment is not to be administered with concomitant administration of dabigatran etexilate mesylate.

In embodiments, the daily dose of Compound A, or a pharmaceutically acceptable salt thereof, is administered once per day (QD).

In embodiments, the daily dose of Compound A, or a pharmaceutically acceptable salt thereof, is administered orally to the subject.

In embodiments, the subject is in a fed state.

In embodiments, the daily dose of Compound A, or a pharmaceutically acceptable salt thereof, is about 200 mg, or an equivalent amount of a pharmaceutically acceptable salt thereof.

In embodiments, the daily dose of Compound A, or a pharmaceutically acceptable salt thereof, is 200 mg or an equivalent amount of a pharmaceutically acceptable salt thereof.

In embodiments, the daily dose of Compound A is about 200 mg.

In embodiments, the daily dose of Compound A is 200 mg.

In embodiments, the daily dose of Compound A, or a pharmaceutically acceptable salt thereof, is about 100 mg or an equivalent amount of a pharmaceutically acceptable salt thereof.

In embodiments, the daily dose of Compound A, or a pharmaceutically acceptable salt thereof, is 100 mg or an equivalent amount of a pharmaceutically acceptable salt thereof.

In embodiments, the daily dose of Compound A is about 100 mg.

In embodiments, the daily dose of Compound A is 100 mg.

In embodiments, Compound A is administered as a free base.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic or locally advanced.

In embodiments, the breast cancer is estrogen receptor positive (ER+) breast cancer.

In embodiments, the estrogen receptor positive (ER+) breast cancer is human epidermal growth factor receptor 2 negative (HER2−).

In embodiments, the subject is human.

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache,

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache; and Compound A, or a pharmaceutically acceptable salt thereof, is administered once per day (QD).

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache; and Compound A, or a pharmaceutically acceptable salt thereof, is orally administered once per day (QD).

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache; and Compound A, or a pharmaceutically acceptable salt thereof, is orally administered as a daily dose of about 200 mg or about 100 mg once per day (QD).

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache; and Compound A, or a pharmaceutically acceptable salt thereof, is orally administered as a daily dose of about 100 mg once per day (QD).

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein Compound A, or a pharmaceutically acceptable salt thereof, is administered once per day (QD).

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein Compound A, or a pharmaceutically acceptable salt thereof, is orally administered as a daily dose of about 200 mg or about 100 mg once per day (QD).

Also disclosed herein is a method for treating cancer in a subject receiving a substrate of P-glycoprotein comprising administering Compound A, or a pharmaceutically acceptable salt thereof, to the subject and monitoring the subject for adverse reactions during treatment with Compound A, or a pharmaceutically acceptable salt thereof, wherein Compound A, or a pharmaceutically acceptable salt thereof, is orally administered as a daily dose of about 100 mg once per day (QD).

Also disclosed herein is a method for treating cancer in a subject receiving dabigatran etexilate mesylate comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush, and headache; and Compound A, or a pharmaceutically acceptable salt thereof, is administered once per day (QD).

Also disclosed herein is a method for treating cancer in a subject receiving dabigatran etexilate mesylate comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache; and Compound A, or a pharmaceutically acceptable salt thereof, is orally administered as a daily dose of about 200 mg or about 100 mg once per day (QD).

Also disclosed herein is a method for treating cancer in a subject receiving dabigatran etexilate mesylate comprising administering Compound A, or a pharmaceutically acceptable salt thereof, to the subject and monitoring the subject for adverse reactions during treatment with Compound A, or a pharmaceutically acceptable salt thereof, wherein the adverse reactions are selected from the group consisting of diarrhea, nausea, dry skin, hot flush and headache; and Compound A, or a pharmaceutically acceptable salt thereof, is orally administered as a daily dose of about 100 mg once per day (QD).

Also disclosed herein is a method for treating cancer in a subject receiving dabigatran etexilate mesylate comprising administering Compound A, or a pharmaceutically acceptable salt thereof, to the subject and monitoring the subject for adverse reactions during treatment with Compound A, or a pharmaceutically acceptable salt thereof, wherein Compound A, or a pharmaceutically acceptable salt thereof, is administered once per day (QD).

Also disclosed herein is a method for treating cancer in a subject receiving dabigatran etexilate mesylate comprising administering Compound A, or a pharmaceutically acceptable salt thereof, to the subject and monitoring the subject for adverse reactions during treatment with Compound A, or a pharmaceutically acceptable salt thereof, wherein Compound A, or a pharmaceutically acceptable salt thereof, is orally administered as a daily dose of about 200 mg or about 100 mg once per day (QD).

Also disclosed herein is a method for treating cancer in a subject receiving dabigatran etexilate mesylate comprising administering Compound A, or a pharmaceutically acceptable salt thereof, to the subject and monitoring the subject for adverse reactions during treatment with Compound A, or a pharmaceutically acceptable salt thereof, wherein Compound A, or a pharmaceutically acceptable salt thereof, is orally administered as a daily dose of about 100 mg once per day (QD).

Also disclosed herein is a method for treating cancer comprising administering to a subject Compound A or a pharmaceutically acceptable salt thereof, wherein Compound A, or a pharmaceutically acceptable salt thereof, is administered once per day (QD) and is not administered concomitantly with a substrate of P-glycoprotein.

Also disclosed herein is a method for treating cancer comprising administering to a subject Compound A or a pharmaceutically acceptable salt thereof, wherein Compound A, or a pharmaceutically acceptable salt thereof, is orally administered once per day (QD) and is not administered concomitantly with a substrate of P-glycoprotein.

Also disclosed herein is a method for treating cancer comprising administering to a subject Compound A or a pharmaceutically acceptable salt thereof, wherein Compound A, or a pharmaceutically acceptable salt thereof, is orally administered as a daily dose of about 200 mg or about 100 mg once per day (QD) and is not administered concomitantly with a substrate of P-glycoprotein.

Also disclosed herein is a method for treating cancer comprising administering to a subject Compound A or a pharmaceutically acceptable salt thereof, wherein Compound A, or a pharmaceutically acceptable salt thereof, is orally administered as a daily dose of about 200 mg or about 100 mg once per day (QD) and is not administered concomitantly with a substrate of P-glycoprotein, and wherein said subject is a human which is in a fed state.

Also disclosed herein is a method for treating cancer comprising administering to a subject Compound A or a pharmaceutically acceptable salt thereof, wherein Compound A, or a pharmaceutically acceptable salt thereof, is administered once per day (QD) and is not administered concomitantly with dabigatran etexilate mesylate.

Also disclosed herein is a method for treating cancer comprising administering to a subject Compound A or a pharmaceutically acceptable salt thereof, wherein Compound A, or a pharmaceutically acceptable salt thereof, is orally administered once per day (QD) and is not administered concomitantly with dabigatran etexilate mesylate.

Also disclosed herein is a method for treating cancer comprising administering to a subject Compound A or a pharmaceutically acceptable salt thereof, wherein Compound A, or a pharmaceutically acceptable salt thereof, is orally administered as a daily dose of about 200 mg or about 100 mg once per day (QD) and is not administered concomitantly with dabigatran etexilate mesylate.

Also disclosed herein is a method for treating cancer comprising administering to a subject Compound A or a pharmaceutically acceptable salt thereof, wherein Compound A, or a pharmaceutically acceptable salt thereof, is orally administered as a daily dose of about 200 mg or about 100 mg once per day (QD) and is not administered concomitantly with dabigatran etexilate mesylate, and wherein said subject is a human which is in a fed state.

In each of the methods described herein, the cancer is preferably breast cancer.

Also disclosed herein is Compound A, or a pharmaceutically acceptable salt thereof, for use according to any one of foregoing embodiments.

Also disclosed herein is Compound A, or a pharmaceutically acceptable salt thereof, for use according to any one of foregoing embodiments.

Also disclosed herein are uses of Compound A, or a pharmaceutically acceptable salt thereof, in the manufacture of a medicament according to any one of foregoing embodiments.

Also disclosed herein are uses of Compound A, or a pharmaceutically acceptable salt thereof, in the manufacture of a medicament according to any one of foregoing embodiments.

Each of the embodiments described herein may be combined with any other embodiment(s) described herein not inconsistent with the embodiment(s) with which it is combined.

### Mode of Administration and Dosing

Concomitant administration means that two or more drugs are given and administered at or almost at the same time (e.g., one after the other, on the same day, etc.). Coadministration means the act of giving or administering (e.g., administration) two or more drugs at the same time

The terms “treat” and “treating” a cancer or a cancer-associated disease, as used herein, mean to administer a therapy according to the present disclosure to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The terms “treatment” and “therapy,” as used herein, unless otherwise indicated, refer to the act of treating as “treating” is defined immediately above. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer may be measured in a number of ways (see, for example, W. A. Weber, J. Nucl. Med. (2009) 50: 1S-10S).

“Fed condition” or “fed state” as used to describe a subject herein, means that the subject has eaten less than 4 hours before a time point of interest, such as the time of administering Compound A. In embodiments, a subject in the fed state has not eaten for at most any of 4, 3, 2, 1, or 0.5 hours prior to administration of Compound A.

As used herein, the terms, “subject”, “participant” and “patient,” are used interchangeably, to refer to a human. Human subjects may be of any gender. In embodiments, a human is an adult human.

An “amount” for use and for treating a subject refers to an amount that provides, in single or multiple doses, a detectable response of any duration of time (transient, medium, or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences, or complications of the disease, to a measurable extent, although reducing or inhibiting a progression or worsening of the disease, or providing stability (i.e., not worsening) state of the disease, is considered a satisfactory outcome. The term “therapeutically effective amount” or “effective amount” also means an amount of an agent that is effective for producing a desired therapeutic effect upon administration to a subject, for example, to stem the growth, or result in the shrinkage, of a cancerous tumor. In reference to the treatment of cancer, a therapeutically effective amount refers to that amount that has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens also may be characterized as the ability to induce, enhance, maintain, or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

Embodiments of the present invention provide a dose, dosage, and dosing regimen comprising administering to a subject an amount, or an effective amount of Compound A, or a pharmaceutically acceptable salt thereof. The amount, the effective amount, or the therapeutically effective amount, may be a daily dose of about 200 mg. In embodiments, a daily dose is 200 mg. In embodiments, the amount, the effective amount, or the therapeutically effective amount, may be a daily dose of about 100 mg. In embodiments, a daily dose is 100 mg.

In embodiments, the daily dose of Compound A, or a pharmaceutically acceptable salt thereof, is administered once per day (QD).

The compounds disclosed herein may be administered orally. Oral administration may involve swallowing, so that the compound enters the gastrointestinal tract, or buccal or sublingual administration may be employed by which the compound enters the bloodstream directly from the mouth.

In embodiments, the daily dose of Compound A, or a pharmaceutically acceptable salt thereof, is administered orally.

Compound A, or a pharmaceutically acceptable salt thereof, may be present in a pharmaceutical composition, which includes at least one pharmaceutically acceptable excipient. A “pharmaceutically acceptable excipient” refers to a component that may be included in the compositions described herein, is physiologically suitable for pharmaceutical use, and causes no significant adverse effects nor therapeutic effects to a subject. The term “excipient” is used herein to describe any ingredient other than the compound(s) of the invention. The choice of excipient will to a large extent depend on factors such as the mode of administration, the effect of the excipient on solubility and stability, and the nature of the dosage form.

The compounds of the methods or uses of the present invention may be formulated prior to administration. The formulation preferably will be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous, and nonaqueous oral solutions and suspensions, packaged in containers adapted for subdivision into individual doses.

Repetition of the administration or dosing regimens may be conducted as necessary to achieve the desired reduction or diminution of cancer cells. A “continuous dosing schedule,” as used herein, is an administration or dosing regimen without dose interruptions, e.g., without days off treatment. Repetition of 28-day treatment cycles without dose interruptions between the treatment cycles is an example of a continuous dosing schedule. In embodiments, the Compound A, or a pharmaceutically acceptable salt thereof, of the present invention may be administered in a continuous dosing schedule. In embodiments, the Compound A, or a pharmaceutically acceptable salt thereof, and a substrate of P-gp disclosed herein may be administered concomitantly in a continuous dosing schedule.

In embodiments, Compound A, or a pharmaceutically acceptable salt thereof, is administered once daily to comprise a complete cycle of 28-days. Repetition of 28-day treatment cycles is continued during treatment in accordance with the methods and uses of the present disclosure.

### Method of Treatment

The term “locally advanced,” as used herein, as it relates to cancer, may or may not be treated with curative intent. For example, locally advanced breast cancer (LABC) is defined by the U.S. National Comprehensive Cancer Network as a subset of breast cancer characterized by the most advanced breast tumors in the absence of distant metastasis, wherein the tumors are more than 5 cm in size with regional lymphadenopathy; tumors of any size with direct extension to the chest wall or skin, or both (including ulcer or satellite nodules), regardless of regional lymphadenopathy; presence of regional lymphadenopathy (clinically fixed or matted axillary lymph nodes, or any of infraclavicular, supraclavicular, or internal mammary lymphadenopathy) regardless of tumor stage. (Garg et al. Curr Oncol. 2015 October; 22(5): e409-10; National Comprehensive Cancer Network NCCN Clinical Practice Guidelines in Oncology: Breast Cancer. Fort Washington, PA: NCCN; 2015. Ver. 2.2015.)

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic or locally advanced.

In embodiments, the breast cancer is estrogen receptor positive (ER+) breast cancer.

In embodiments, the estrogen receptor positive (ER+) breast cancer is human epidermal growth factor receptor 2 negative (HER2−).

### Example

In order that this invention may be better understood, the following example is set forth. This example is for purposes of illustration only and is not to be construed as limiting the scope of the invention in any manner.

### Abbreviations

AUC_{inf}=area under the plasma concentration-time curve from time 0 extrapolated to infinite time;
AUCₗₐₛₜ=area under the plasma concentration-time profile from time 0 to the time of the last quantifiable concentration (Cₗₐₛₜ);
Cₘₐₓ=maximum plasma concentration;
CI=confidence interval;
hrs=hours;
ECG=electrocardiogram;
mins=minutes;
PE=physical examination;
TEAE=treatment emergent adverse event

### A Study to Understand the Effect of Compound a on Dabigatran Etexilate Pharmacokinetics (PK) in Healthy Adults

### Overview

Compound A was investigated in an interventional, Phase 1, open-label, fixed sequence, 2-period study to evaluate the effect of a single oral dose of Compound A on the PK of dabigatran in healthy participants.

### Objective

The objective of this drug-drug interaction study was to evaluate the impact of Compound A on the PK of dabigatran etexilate (a model substrate for P-gp) in healthy adults (NCT05673889)

### Brief Plain Language Summary

The purpose of this study was to understand if Compound A affected how a medicine called dabigatran etexilate was absorbed or processed into the body in healthy adults.

All participants in this study received one dose of dabigatran etexilate mesylate alone by mouth in Period 1. In Period 2, everyone received one dose of dabigatran etexilate mesylate by mouth approximately 90 mins after receiving one dose of Compound A by mouth. The levels of dabigatran in Period I were compared to the levels of dabigatran in Period 2. This study design was used to determine if and how Compound A affects dabigatran absorption into the body in healthy adults.

All participants stayed at the study clinic for approximately 8 days and 7 nights.

### Methodology

This was a Phase 1, open-label, 2-period, fixed-sequence study to estimate the effect of a single oral dose of Compound A on the PK of a P-gp substrate, dabigatran etexilate (as mesylate), in healthy male participants and female participants with nonchildbearing potential.

This study consisted of 2 periods with a fixed sequence treatment design.

On Period 1 Day 1, a standard breakfast (approximately 700 calories with a fat content of approximately 35%) was provided prior to dosing. The standard breakfast was required to be completely consumed within an approximately 20-minute period. A single dose of 75 mg dabigatran etexilate (as mesylate) (as 1 capsule of 75 mg) was administered approximately 2 hrs (120 mins) after starting the standard breakfast. To adequately remove any drug effects of dabigatran, a minimum washout period of 4 days was required after dabigatran etexilate (as mesylate) administration on Period 1 Day 1. Serial PK samples were collected up to 48 hrs after single dose administration to determine dabigatran PK parameters (total).

On Period 2 Day 1, a standard breakfast (approximately 700 calories with a fat content of approximately 35%) was provided prior to Compound A and dabigatran etexilate (as mesylate) dosing. The standard breakfast was required to be the same as provided in Period 1 Day 1 and was required to be completely consumed within an approximately 20-minute period. A single dose of 200 mg Compound A (as 2 tablets of 100 mg) was administered approximately 10 mins after the completion of the breakfast. A single dose of 75 mg dabigatran etexilate

(as mesylate) (as 1 capsule of 75 mg) was administered approximately 1.5 hour (90 mins) after the start of Compound A dosing, which was approximately 2 hrs (120 mins) after the start of the breakfast. Serial PK samples were collected up to 48 hrs post dabigatran etexilate (as mesylate) dosing to determine the single dose PK parameters of Compound A and dabigatran (total)
Masking: No masking (open label)
Enrollment number: 24

### Objectives and Endpoints

The objectives and endpoints of the clinical study are shown below in Table 1.

TABLE 1

| | | |
|---|---|---|
| Type | Objectives | Endpoints |

Primary:

| | | |
|---|---|---|
| PK | To estimate the effect of a | Plasma total dabigatran (sum of |
| | single oral 200 mg dose of | unconjugated and glucuronide- |
| | Compound A on the single | conjugated dabigatran) PK |
| | dose pharmacokinetic of | parameters alone and |
| | dabigatran in healthy | following administration of a |
| | participants. | single 200 mg dose of Compound |
| | | A: Cₘₐₓ, AUC_{inf} (AUCₗₐₛₜ if AUC_{inf} |
| | | cannot be estimated). |

Secondary:

| | | |
|---|---|---|
| Safety | To evaluate the safety and | TEAEs, clinical laboratory tests, |
| | tolerability of dabigatran | vital signs, PE, and ECGs. |
| | etexilate (as mesylate) | |
| | alone and following | |
| | administration with a | |
| | single 200 mg dose of | |
| | Compound A. | |

### Statistical Methods

Natural log transformed parameters (AUC_{inf} [if data permitted], AUCₗₐₛₜ, and Cₘₐₓ) of total dabigatran were analyzed using a mixed effect model with treatment as a fixed effect and participant as a random effect. Note that area under the concentration-time curve from time zero to 24 hrs (AUC₂₄) instead of AUCₗₐₛₜ was used for the statistical analysis due to varied times of the last measurable concentration across participants. Estimates of the adjusted mean differences (Test-Reference) and corresponding 90% CIs were obtained from the model. The adjusted mean differences and 90% CIs for the differences were exponentiated to provide estimates of the ratio of adjusted geometric means (Test/Reference) and 90% CI for the ratios. Dabigatran etexilate mesylate administered alone was the reference treatment and

Compound A co-administered with dabigatran etexilate mesylate was the test treatment. All safety analyses were performed on the safety analysis set. Safety data were presented in tabular and/or graphical format and summarized descriptively, where appropriate in accordance with the Clinical Data Interchange Standards Consortium Standards (CaPS).

### Interventions

The single arm of the clinical study is shown below in Table 2.

TABLE 2

| Arm title | Type | Description |
|---|---|---|
| Dabigatran | Experimental | Dabigatran etexilate |
| etexilate | | mesylate administered as a |
| with/without | | single dose in Period 1 and |
| Compound A | | Period 2. Compound A |
| | | administered as a single |
| | | dose in Period 2. |

The drug interventions explored in the study are shown in Table 3.

TABLE 3

| Intervention | | Associated | | |
|---|---|---|---|---|
| Name | Type | Arms | Description | Other Names |
| Compound A | Drug | Dabigatran | Experimental | PF-07850327; |
| | | etexilate | | ARV-471 |
| | | with/without | | |
| | | Compound A | | |
| Dabigatran | Drug | Dabigatran | Probe | PRADAXA ® |
| etexilate | | etexilate | substrate | |
| mesylate | | with/without | | |
| | | Compound A | | |

The outcome measures in the study are shown in Table 4.

TABLE 4

| Outcome Measure | Timeframe | Description |
|---|---|---|
| Maximum observed | Period 1- Day 1 Pre-dose, | |
| concentration (Cₘₐₓ) of | 0.5, 1, 2, 3, 4, 6, 8, 12, 24, | |
| dabigatran¹ when dabigatran | 36, 48 hrs post-dose | |
| etexilate mesylate is | | |
| administered alone | | |
| Maximum observed | Period 2 - Day 1 pre-dose, | |
| concentration (Cₘₐₓ) of | 0.5, 1, 2, 3, 4, 6, 8, 12, 24, | |
| dabigatran¹ when dabigatran | 36, 48 hrs post-dose | |
| etexilate mesylate is | | |
| administered after | | |
| Compound A | | |
| Arca under the curve from | Period 1- Day 1 Pre-dose, | AUC (0-∞) = Area under |
| time zero to extrapolated | 0.5, 1, 2, 3, 4, 6, 8, 12, 24, | the plasma concentration |
| infinite time [AUC (0-∞)] | 36, 48 hrs post-dose | versus time curve (AUC) |
| of dabigatran¹ when | | from time |
| dabigatran etexilate | | zero (pre-dose) to |
| mesylate is administered | | extrapolated infinite time |
| alone | | (0-∞). It is obtained from |
| | | AUC (0-t) plus AUC (t-∞). |
| Area under the curve from | Period 2 - Day 1 pre-dose, | AUC (0-∞) = Area under |
| time zero to extrapolated | 0.5, 1, 2, 3, 4, 6, 8, 12, 24, | the plasma concentration |
| infinite time [AUC (0-∞)] | 36, 48 hrs post-dose | versus time curve (AUC) |
| of dabigatran¹ when | | from time zero (pre-dose) to |
| dabigatran etexilate | | extrapolated infinite time |
| mesylate is administered | | (0-∞). It is obtained from |
| after Compound A | | AUC (0-t) plus AUC (t-∞). |

¹When referring to pharmacokinetic results, dabigatran refers to the sum of unconjugated and glucuronide-conjugated dabigatran.

¹When referring to pharmacokinetic results, dabigatran refers to the sum of unconjugated and glucuronide-conjugated dabigatran.

The secondary outcome measures in the study are shown in Table 5.

TABLE 5

| Outcome Measure | Timeframe | Description |
|---|---|---|
| Number of participants with | Time the participant | An AB is any untoward |
| treatment-emergent adverse | provides informed consent | medical occurrence in a |
| events (AEs) and serious | through and including | participant who received |
| adverse events (SAEs) | follow-up contact occurring | study drug without regard to |
| | 28 to 35 calendar days after | possibility of causal |
| | the last administration of the | relationship with the study |
| | study intervention. | treatment. SAE is defined as |
| | | one of the following: is fatal |
| | | or life-threatening; results in |
| | | persistent or significant |
| | | disability/incapacity; |
| | | constitutes a congenital |
| | | anomaly/birth defect; is |
| | | medically significant; |
| | | requires inpatient |
| | | hospitalization or |
| | | prolongation of existing |
| | | hospitalization. Treatment- |
| | | emergent AE is defined as |
| | | an AE with onset date |
| | | occurring during the on- |
| | | treatment period. AEs |
| | | include all SAEs and non- |
| | | SAEs. |
| Number of participants with | Baseline up to Period 2 Day | Following parameters were |
| clinical laboratory | 3 | analyzed for laboratory |
| abnormalities | | examination: hematology |
| | | (hemoglobin, hematocrit, red |
| | | blood cell count, platelet |
| | | count, white blood cell |
| | | count, total neutrophils, |
| | | cosinophils, monocytes, |
| | | basophils, lymphocytes); |
| | | liver function (aspartate |
| | | aminotransferase, alanine |
| | | aminotransferase, total |
| | | bilirubin, alkaline |
| | | phosphatase, albumin, total |
| | | protein); renal function |
| | | (blood urea nitrogen, |
| | | creatinine, uric acid, |
| | | cystatinC); electrolytes |
| | | (sodium, potassium, |
| | | chloride, calcium, |
| | | bicarbonate); clinical |
| | | chemistry (glucose); |
| | | urinalysis (dipstick [decimal |
| | | logarithm of reciprocal of |
| | | hydrogen ion activity {pH} |
| | | of urine, glucose, protein, |
| | | blood, ketones, nitrites, |
| | | leukocyte esterase, |
| | | urobilinogen, bilirubin], |
| | | microscopy. |
| Number of participants with | Baseline up to Period 2 Day | ECG abnormalities criteria |
| electrocardiogram (ECG) | 3 | included a) a postdose QTcF |
| abnormalities | | is increased by >60 ms from |
| | | the baseline and is >450 ms; |
| | | or b) an absolute QTcF value |
| | | is >500 ms for any |
| | | scheduled ECG. |
| Number of participants with | Baseline up to Period 2 Day | Blood pressure and pulse |
| clinically significant change | 3 | rate was performed |
| from baseline in vital signs | | following at least a 5-minute |
| | | rest in a supine position. |

Study eligibility is shown in Table 6.

TABLE 6

| | |
|---|---|
| Age | All |
| Age Limits | Min. Age: 18 |
| | Max. Age: 70 years |
| Accepts Healthy Volunteers | Yes |
| Eligibility Criteria | Inclusion Criteria: - Healthy male and/or |
| | female participants of non-childbearing |
| | potential who were overtly healthy as |
| | determined by medical evaluation and were |
| | between the ages of 18 and 70 years, |
| | inclusive at the time of signing the informed |
| | consent document (ICD). - Body mass index |
| | (BMI) of 17.5 to 30.5 kg/m\hat{ }2; and a total |
| | body weight >50 kg (110 lb).- Participants |
| | who were willing and able to comply with |
| | all scheduled visits, treatment plan, |
| | laboratory tests, and other study procedures. |
| | Exclusion Criteria: - Evidence or history of |
| | clinically significant hematological, renal, |
| | endocrine, pulmonary, gastrointestinal, |
| | cardiovascular, hepatic, psychiatric, |
| | neurological, or allergic disease (including |
| | drug allergies, but excluding untreated, |
| | asymptomatic, seasonal allergies at the time |
| | of dosing).- Pregnant female participants, |
| | breastfeeding female participants, female |
| | participants of childbearing potential. Male |
| | participants with partners currently |
| | pregnant; male participants of childbearing |
| | potential who were unwilling or unable to |
| | use a highly effective method of |
| | contraception as outlined in this protocol for |
| | the duration of the study and for 90 days |
| | after the last dose of investigational |
| | product.- Active bleeding or risk of bleeding |
| | including prior personal or familiar history |
| | of abnormal bleeding, hereditary or acquired |
| | coagulation or platelet disorder or abnormal |
| | coagulation test (PT/INR or PTT/aPTT |
| | greater than upper limit of normal) result at |
| | screening. Any significant risk factor for |
| | major bleeding, which may include but not |
| | limited to current or recent GI ulceration, |
| | presence of malignant neoplasms at high |
| | risk of bleeding, recent brain or spinal |
| | injury, recent brain, spinal or ophthalmic |
| | surgery, recent intracranial hemorrhage, |
| | known or suspected esophageal varices, |
| | arteriovenous malformations, vascular |
| | aneurysms or major intraspinal or |
| | intracerebral vascular abnormalities. Other |
| | medical or psychiatric condition including |
| | recent (within the past year) or active |
| | suicidal ideation/behavior or laboratory |
| | abnormality, or other conditions or |
| | situations related to COVID-19 pandemic |
| | that may increase the risk of study |
| | participation or, in the investigator's |
| | judgment, make the participant |
| | inappropriate for the study. |
| | - Use of prescription or nonprescription |
| | medications, including vitamins, dietary and |
| | herbal supplements, grapefruit/grapefruit |
| | containing products, and Seville |
| | orange/Seville orange containing products |
| | within 7 days or 5 half-lives (whichever is |
| | longer) prior to the first dose of study |
| | intervention. A longer washout is required |
| | for those that fall into the categories below: |
| | --Moderate or strong cytochrome P450 |
| | (CYP) 3A/P-gp inducers which were |
| | prohibited within 14 days plus 5 half-lives |
| | prior to the first dose of study intervention. |
| | --Moderate or strong CYP3A/P-gp |
| | inhibitors which were prohibited within |
| | 14 days or 5 half-lives (whichever is longer) |
| | prior to the first dose of study |
| | intervention. |
| | - Standard 12-lead electrocardiogram (ECG) |
| | that demonstrated clinically relevant |
| | abnormalities that may affect participant |
| | safety or interpretation of study results. |
| | - History of sensitivity to heparin or heparin- |
| | induced thrombocytopenia. |

### Safety Results

A total of 9 all-causality adverse events (AEs) were reported in 6 (25.0%) participants following a single dose of dabigatran etexilate mesylate 75 mg (Treatment Period 1), of which, 4 AEs reported in 3 (12.5%) participants were considered as treatment related. A total of 7 all-causality AEs were reported in 6 (25.0%) participants following a single dose of Compound A 200 mg and dabigatran etexilate mesylate 75 mg (Treatment Period 2), of which, 1 AE reported in 1 (4.2%) participant was considered treatment related. No serious adverse events (SAEs), severe AEs, discontinuations or dose reductions due to AEs were reported in this study.

Following a single dose of dabigatran etexilate mesylate 75 mg, mild AEs of diarrhea, lip dry, nausea, dry throat, dry skin and hot flush were reported in 1 (4.2%) participant each, of which, diarrhea, nausea, dry skin and hot flush were considered as treatment related. Moderate AEs of hematoma and myalgia were reported in 2 (8.3%) and 1 (4.2%) participants respectively, which were not considered as treatment related. Following a single dose of Compound A 200 mg and dabigatran etexilate mesylate 75 mg, mild AEs of influenza, contusion, wound, oropharyngeal pain and rhinorrhea were reported in 1 (4.2%) participant each, which were not considered as treatment related. Moderate AEs of contusion and headache were reported in 1 (4.2%) participant each, of which, headache was considered as treatment related.

Following a single dose of dabigatran etexilate mesylate 75 mg, 2 (8.3%) participants had Monocytes/Leukocytes >1.2× upper limit of normal (ULN). Following a single dose of Compound A 200 mg and dabigatran etexilate mesylate 75 mg, 2 (8.3%) participants had Monocytes/Leukocytes >1.2×ULN, 1 (4.2%) participant had Leukocyte Esterase ≥1.

No participants bad laboratory abnormalities that met the predefined criteria for clinical chemistry analyses. No laboratory abnormalities were reported as AEs by the investigator or the sponsor.

No clinically meaningful changes in vital signs measurements, ECGs or other observations related to safety were observed in this study.

### Safety Conclusions

All study interventions were safe and well-tolerated in healthy adult participants. No new safety issues were identified. All AEs were mild or moderate. No serious or severe AEs occurred and no discontinuations or dose reductions/modifications due to AEs were reported.

### Pharmacokinetic Results

### Dabigatran (Total) Pharmacokinetics

Co-administration with a single dose of Compound A (200 mg) increased dabigatran (75 mg) plasma exposure AUC_{inf} by 98% and Cₘₐₓ by 92%.

The ratios of the adjusted geometric means (90% CI) for dabigatran AUC_{inf}, AUC₂₄ and Cₘₐₓ were 197.81% (177.32%, 220.66%), 209.42% (185.03%, 237.03%), and 192.20% (166.56%, 221.79%), respectively, following dabigatran coadministration with Compound A (Test) as compared to administration alone (Reference).

### Summary of Results

A total of 24 participants were enrolled and treated in this study. Co-administration of dabigatran with Compound A increased the area under the plasma concentration-time curve from the time of dosing (time 0) extrapolated to infinite time (AUC_{inf}) and the maximum plasma concentration (Cₘₐₓ) of dabigatran by 98% and 92%, respectively (primary endpoints). The ratios of the adjusted geometric means (90% CI) for dabigatran AUC_{inf} and Cₘₐₓ were 197.81% (177.32%-220.66%) and 192.20% (166.56%-221.79%), respectively, following dabigatran administration with Compound A (test) compared with administration alone (reference). After dabigatran treatment and after co-administration of dabigatran and Compound A, treatment-emergent adverse events (TEAEs) occurred in 6 (25.0%) participants each. Treatment-related AEs occurred in 3 (12.5%) participants after dabigatran treatment (diarrhea, dry skin, hot flush, and nausea; 1 [4.2%] each) and in 1 (4.2%) participant after co-administration of dabigatran and Compound A (headache). All AEs were mild or moderate; no serious or severe AEs occurred and no discontinuations or dose modifications due to AEs were reported.

### CONCLUSIONS

Without wishing to being bound by theory, co-administration of Compound A increased dabigatran exposure, indicating that Compound A is a P-gp inhibitor. Caution is recommended when administering Compound A with sensitive substrates of P-gp, such as dabigatran, to cancer patients.

We claim:

1. A method for treating cancer in a subject receiving a substrate of P-glycoprotein comprising administering Compound A having a structure of: 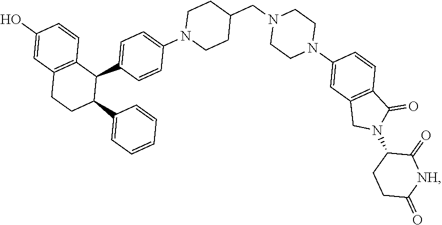 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, to the subject and monitoring the subject for adverse reactions during treatment with Compound A, or a pharmaceutically acceptable salt thereof.

2. A method for treating cancer comprising administering Compound A having a 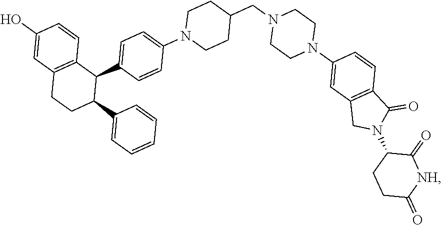 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, to a subject, wherein a physician is provided with instructions to monitor the subject for adverse reactions during concomitant administration of Compound A, or a pharmaceutically acceptable salt thereof, and a substrate of P-glycoprotein.

3. The method of claim 1 or 2, wherein the adverse reactions are selected from the group consisting of diarrhea, nausea, dry skin, hot flush, and headache.

4. The method of any one of claims 1 to 3, wherein a daily dose of Compound A, or a pharmaceutically acceptable salt thereof, is administered to the subject.

5. The method of claim 4, wherein the daily dose of Compound A, or a pharmaceutically acceptable salt thereof, is 200 mg.

6. The method of claim 5, wherein the daily dose of Compound A, or a pharmaceutically acceptable salt thereof, is reduced to 100 mg.

7. The method of any one of claims 4 to 6, wherein the daily dose of Compound A, or a pharmaceutically acceptable salt thereof, is administered once per day.

8. The method of any one of claims 4 to 7, wherein the daily dose of Compound A, or a pharmaceutically acceptable salt thereof, is administered orally to the subject.

9. The method of any one of claims 1 to 8, wherein the subject is in a fed state.

10. The method of claim 3, wherein the daily dose of Compound A, or a pharmaceutically acceptable salt thereof, is not to be administered with concomitant administration of a substrate of P-glycoprotein.

11. The method of claim 1, 2, or 10, wherein the substrate of P-glycoprotein is apixaban, colchicine, cyclosporine, dabigatran etexilate mesylate, digoxin, edoxaban, fexofenadine, rivaroxaban, tacrolimus, or talinolol.

12. The method of claim 11, wherein the substrate of P-glycoprotein is dabigatran etexilate mesylate.

13. The method of any one of claims 1 to 12, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

14. The method of claim 13, wherein the cancer is breast cancer, lung cancer, prostate cancer, pancreatic cancer, or ovarian cancer.

15. The method of claim 14, wherein the cancer is breast cancer, lung cancer, or prostate cancer.

16. The method of claim 15, wherein the cancer is breast cancer.

17. The method of claim 16, wherein the breast cancer is metastatic or locally advanced.

18. The method of claim 16 or 17, wherein the breast cancer is estrogen receptor positive (ER+) breast cancer.

19. The method of claim 18, wherein the estrogen receptor positive (ER+) breast cancer is human epidermal growth factor receptor 2 negative (HER2−).

20. The method of any one of claims 1 to 19, wherein the subject is human.
